(19) United States
(12) Patent Application Publication
(10) Pub. No.: US 2014/0227198 A1
(43) Pub. Date: Aug. 14, 2014

(54) SHELF-STABLE OLIVE EXTRACT-CONTAINING COMPOSITIONS AND METHODS OF USE THEREOF

(71) Applicant: GRADS Holdings LLLP, Atlanta, GA (US)

(72) Inventor: John Alkire, Atlanta, GA (US)

(73) Assignee: GRADS Holdings LLLP, Atlanta, GA (US)

(21) Appl. No.: 14/176,884

(22) Filed: Feb. 10, 2014

Related U.S. Application Data
(60) Provisional application No. 61/764,240, filed on Feb. 13, 2013.
(60) Provisional application No. 61/911,158, filed on Dec. 3, 2013.

(51) Int. Cl. A61K 36/63; A61K 45/06; A23L 1/30

(52) U.S. Cl. CPC A61K 36/63; A23L 1/3002; A61K 45/06
USPC 424/43; 424/769; 424/766; 424/754; 424/715

(57) ABSTRACT

Shelf stable compositions comprising olive extracts are described herein. The compositions include an olive extract comprising hydroxytyrosol, tyrosol, and oleuropein, wherein the weight ratio of hydroxytyrosol to oleuropein is 3:1 or less and the weight ratio of hydroxytyrosol to tyrosol is 2.85:1 or less. The compositions are stable for at least 12 months. Methods of using the shelf stable compositions are also described herein.

### CROSS-REFERENCE TO PRIORITY APPLICATIONS

This application claims priority to U.S. Provisional Application No. 61/764,240, filed Feb. 13, 2013, and U.S. Provisional Application No. 61/911,158, filed Dec. 3, 2013, which are incorporated herein by reference in their entireties.

### BACKGROUND

Olive polyphenols are highly unstable compounds when hydroxytyrosol, tyrosol, and oleuropein are each present. When stored, the profile of the polyphenols changes dramatically over time. For example, a 70% or greater reduction in olive polyphenols can result over a 12 month period, as well as dramatic ratio changes of the polyphenols.

### SUMMARY

Provided herein are olive extract-containing compositions useful for promoting joint health in a subject. A composition described herein includes an olive extract comprising hydroxytyrosol, tyrosol, and oleuropein, wherein the weight ratio of hydroxytyrosol to oleuropein is 3:1 or less and the weight ratio of hydroxytyrosol to tyrosol is 2.85:1 or less. The compositions described herein are stable for at least twelve months at ambient temperature. Optionally, at least 80% of the oleuropein in the olive extract is present after twelve months of storage (e.g., at least 85% or at least 90% of the oleuropein in the olive extract is present after twelve months of storage at ambient temperature.

The compositions can further comprise at least one additional compound selected from the group consisting of a grape extract, quercetin, red onion powder or extract, naringin, sinomenine, piperine, glycyrrhizin, a nitrile glycoside, cuminum cyminum, curcumin, vitamin D, willow bark extract, theobromine, a coffee extract, and a hydrogen carbonate.

Optionally, the weight ratio of hydroxytyrosol to tyrosol is from 1:1 to 2.85:1 (e.g., from 1.5:1 to 2.75:1 or from 2:1 to 2.7:1). The weight ratio of hydroxytyrosol to oleuropein can be from 1:1 to 3:1 (e.g., from 1.5:1 to 2.8:1 or from 2:1 to 2.7:1). Optionally, the weight ratio of tyrosol to oleuropein can be from 1:1 to 2.5:1 (e.g., from 1:1 to 2:1).

The olive extract can be in the form of a powder or a liquid. The composition can be suitable for oral administration. Optionally, the composition is a nutraceutical composition or a dietary supplement. Optionally, the composition is in the form of a beverage, nutritional bar, food additive, tablet, pill, oil, granule, capsule, or effervescent formulation.

Also provided herein are methods of maintaining health and comfort in a subject. The methods of maintaining health and comfort in a subject comprise selecting a healthy subject (e.g., a non-arthritic subject or a non-diseased subject) and administering to the subject a composition as described herein. Optionally, the composition is administered to the subject orally. For example, the olive extract can be administered to the subject in an amount of from 1 mg to 2000 mg per day. The methods can further comprise administering to the subject a second compound or composition, wherein the second compound or composition includes a nutraceutical additive, a vitamin, a mineral, or an herbal extract.

Further provided herein is a use of a composition as described herein for maintaining and supporting health.

Also provided herein is a dietary supplement comprising a composition comprising an olive extract comprising hydroxytyrosol, tyrosol, and oleuropein, wherein the weight ratio of hydroxytyrosol to oleuropein is 3:1 or less and the weight ratio of hydroxytyrosol to tyrosol is 2.85:1 or less. The composition is stable for at least twelve months at ambient temperature.

The details of one or more embodiments are set forth in the accompanying description below. Other features, objects, and advantages will be apparent from the description and from the claims.

### DETAILED DESCRIPTION

Described herein are storage stable olive extract compositions containing hydroxytyrosol, oleuoropein, and tyrosol. The compositions are useful in promoting health in a subject (e.g., in a healthy subject). Also provided herein are methods for their use in promoting joint health, energy, cardiovascular health, and immune health in subjects. In these methods, a healthy subject is selected and the compositions are administered to the subject.

I. Compositions

The compositions described herein are useful for supporting or improving health and comfort in subjects (e.g., healthy subjects). For example, the compositions are useful in promoting joint health, energy, cardiovascular health, and immune health in subjects. The composition can be a purified composition that includes an olive extract. The olive extract described herein includes one or more phenolic compounds, including hydroxytyrosol, oleuropein, and tyrosol. Optionally, the phenolic components are obtained from a purified form of olive extract. The olive extracts described herein can be obtained from fruit, leaves, oil, or olive water from the olive tree (i.e., Olea europaea). The olive extract containing the hydroxytyrosol, tyrosol, and oleuropein can be in the form of a liquid or liquid drops. The olive extract containing the hydroxytyrosol, tyrosol, and oleuropein can also be dried to provide a powder extract. Optionally, the olive leaf or fruit can be incorporated into the composition in the form of a liquid concentrate, a dried leaf tea, a powder, a capsule, a tablet, a pill, and other forms.

Hydroxytyrosol (i.e., 3,4-dihydrophenethyl alcohol) is a phenolic phytochemical that has antioxidant properties. In pure form, hydroxytyrosol is a colorless, odorless liquid. Olives, leaves, and olive pulp contain large amounts of hydroxytyrosol (as compared to olive oil) and most of the substance can be recovered from these sources to produce hydroxytyrosol extracts. Optionally, hydroxytyrosol can comprise 15% or less of the dry weight of the olive extract (i.e., weight of the olive extract without any aqueous solvent or carrier). For example, hydroxytyrosol can be present in the olive extract in an amount of 14% or less, 13% or less, 12% or less, 11% or less, 10% or less, 9% or less, 8% or less, 7% or less, 6% or less, or 5% or less based on the dry weight of the olive extract. Hydroxytyrosol can optionally be present in an amount of from 9% to 11% based on the dry weight of the olive extract (e.g., 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, or 10.9% based on the dry weight of the olive extract).

Oleuropein is a phenolic compound found in the olive leaf from the olive tree. Oleuropein is a tyrosol ester of elenolic acid that is further hydroxylated and glycosylated. Similar to hydroxytyrosol, oleuropein has a powerful antioxidant activity both in vivo and in vitro. Optionally, oleuropein can comprise 10% or less of the dry weight of the olive extract. For example, oleuropein can be present in the olive extract in an amount of 9% or less, 8% or less, 7% or less, 6% or less, 5% or less, 4% or less, 3% or less, 2% or less, or 1% or less based on the dry weight of the olive extract. Oleuropein can optionally be present in an amount of from 3% to 5% based on the dry weight of the olive extract (e.g., 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, or 4.9% based on the dry weight of the olive extract).

Tyrosol (i.e., 4-hydroxyphenethyl alcohol) is a phenolic compound that is present in olives and olive oil. Tyrosol also exhibits antioxidant activity in in vivo studies. Optionally, tyrosol can comprise 10% or less of the dry weight of the olive extract. For example, tyrosol can be present in the olive extract in an amount of 9% or less, 8% or less, 7% or less, 6% or less, 5% or less, 4% or less, 3% or less, 2% or less, or 1% or less based on the dry weight of the olive extract. Tyrosol can optionally be present in an amount of from 2% to 4% based on the dry weight of the olive extract (e.g., 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, or 3.9% based on the dry weight of the olive extract).

In the compositions described herein, the weight ratio of hydroxytyrosol to oleuropein can be less than 3:1 (e.g., less than 2.9:1, less than 2.8:1, less than 2.7:1, less than 2.6:1, less than 2.5:1, less than 2.4:1, less than 2.3:1, less than 2.2:1, less than 2.1:1, less than 2.0:1, less than 1.9:1, less than 1.8:1, less than 1.7:1, less than 1.6:1, less than 1.5:1, less than 1.4:1, less than 1.3:1, less than 1.2:1, less than 1.1:1, less than 1.0:1, less than 0.9:1, less than 0.8:1, less than 0.7:1, less than 0.6:1, less than 0.5:1, less than 0.4:1, less than 0.3:1, less than 0.2:1, or less than 0.1:1).

Optionally, the weight ratio of hydroxytyrosol to tyrosol is from 1:1 to 2.85:1 (e.g., from 1.1:1 to 2.8:1, from 1.2:1 to 2.7:1, from 1.3:1 to 2.6:1, from 1.4:1 to 2.5:1, from 1.5:1 to 2.4:1, from 1.6:1 to 2.3:1, from 1.7:1 to 2.2:1, from 1.8:1 to 2.1:1, from 1.9:1 to 2.0:1). For example, the weight ratio of hydroxytyrosol to tyrosol can be from 1.5:1 to 2.75:1 or from 2:1 to 2.7:1.

The weight ratio of hydroxytyrosol to oleuropein can be from 1:1 to 3:1 (e.g., from 1.1:1 to 2.9:1, from 1.2:1 to 2.8:1, from 1.3:1 to 2.7:1, from 1.4:1 to 2.6:1, from 1.5:1 to 2.5:1, from 1.6:1 to 2.4:1, from 1.7:1 to 2.3:1, from 1.8:1 to 2.2:1, or from 1.9:1 to 2.1:1). Optionally, the weight ratio of hydroxytyrosol to oleuropein can be from 1.5:1 to 2.8:1 or from 2:1 to 2.7:1.

The weight ratio of tyrosol to oleuropein can be from 1:1 to 2.5:1 (e.g., from 1.1:1 to 2.4:1, from 1.2:1 to 2.3:1, from 1.3:1 to 2.2:1, from 1.4:1 to 2.1:1, from 1.5:1 to 2:1, or from 1.6:1 to 1.9:1). Optionally, the weight ratio of tyrosol to oleuropein can be from 1:1 to 2:1.

Optionally, the compositions described herein can further include at least one additional compound. The at least one additional compound optionally can include extracts of grapes, quercetin, red onion powder or extract, naringin, sinomenine, piperine, glycyrrhizin, a nitrile glycoside, cuminum cyminum, curcumin, vitamin D, willow bark extract, theobromine, coffee extracts, or a hydrogen carbonate. The combinations of the at least one additional compound and the compositions described herein can have positive effects for supporting or improving health and comfort in healthy subjects, as well as many other benefits as described herein.

Quercetin, a flavonol, is a plant-derived flavonoid found in fruits, vegetables, leaves, and grains. It can be used as a component in the compositions. Optionally, quercetin can be used in the composition when the composition takes the form of a supplement, beverage, or food. Red onion powder or extract, sometimes referred to as purple onions, are cultivars of the onion with purplish red skin and white flesh tinged with red. Powders and extracts obtained from these onions can be included in the compositions described herein. Naringin is a flavanone glycoside that is found, for example, in citrus fruits such as grapefruit. Naringin can be included in the compositions described herein. Sinomenine is an alkaloid found in the root of the climbing plant Sinomenium acutum, which is native to Japan and China. This compound can be included in the compositions described herein.

Piperine is the alkaloid responsible for the pungency of black pepper and long pepper and can be used in the compositions described herein. Glycyrrhizin (i.e., glycyrrhizic acid or glycyrrhizinic acid) is the main sweet-tasting compound from liquorice root. This compound can be incorporated in the compositions described herein.

A nitrile glycoside is a compound that can be extracted from Moringa oleifera. Moringa oleifera (i.e., Moringa pterygosperma) is the most widely cultivated species of the genus Moringa. English common names include moringa and drumstick tree. One or more nitrile glycosides can be incorporated in the compositions described herein. Cuminum cyminum Linn (i.e., cuminum cyminum) is a flowering plant in the family Apiaceae, native from the east Mediterranean to India. Cumin is the dried seed of the herb Cuminum cyminum. Cuminum cyminum and its components can be incorporated in the compositions described herein. Curcumin is the principal curcuminoid of the popular Indian spice turmeric, which is a member of the ginger family (i.e., Zingiberaceae). Curcumin can exist in several tautomeric forms, including a 1,3-diketo form and two equivalent enol forms. Curcumin can be included in the compositions described herein.

Vitamin D encompasses a group of fat-soluble secosteroids responsible for intestinal absorption of calcium and phosphate. In humans, vitamin D can be ingested as cholecalciferol (vitamin D₃) or ergocalciferol (vitamin D₂). In addition, the human body can also synthesize it from cholesterol when sun exposure is adequate (hence its nickname, the “sunshine vitamin”). Any form of vitamin D (e.g., vitamin D₁, vitamin D₂, vitamin D₃, vitamin D₄, or vitamin D₅) can be included in the compositions described herein. Willow bark extract is extracted from willows, sallows, and osiers from the genus Salix. Willow bark extract can be included in the compositions described herein.

Caffeine is a bitter, white crystalline xanthine alkaloid that acts as a stimulant drug. Caffeine is found in varying quantities in the seeds, leaves, and fruit of some plants, where it acts as a natural pesticide that paralyzes and kills certain insects feeding on the plants. It is most commonly consumed by humans in infusions extracted from the seed of the coffee plant and the leaves of the tea bush, as well as from various foods and drinks containing products derived from the kola nut. Other sources include yerba maté, guarana berries, guayusa, and the yaupon holly. Caffeine can be included in the compositions described herein. Bicarbonate (i.e., hydrogen carbonate) is an anion with the chemical formula HCO₃⁻. The most common salt of the bicarbonate ion is sodium bicarbonate, NaHCO₃, which is commonly known as baking soda. Bicarbonates can be included in the compositions described herein.

Optionally, one or more of the components of the compositions described herein are obtained from a natural source, such as, for example, plants, vegetables, herbs, or spices. Optionally, the composition components can be obtained from any other natural source or process, such as by fermentation. Optionally, the composition components can be formulated using synthetic means from a variety of compound sources.

The compositions described herein can further include a liquid carrier. Suitable liquid carriers can be aqueous or non-aqueous carriers. Examples of suitable non-aqueous carriers include propylene glycol, polyethylene glycol, and oils, including those of petroleum, animal, vegetable or synthetic origin, such as peanut oil, soybean oil, mineral oil, sesame oil, olive oil, and the like. Organic esters such as ethyl oleate are also suitable non-aqueous carriers. Aqueous carriers include water, ethanol, glycerol, alcoholic/aqueous solutions, emulsions, or suspensions, including saline and buffered media. The composition, if desired, can also contain wetting or emulsifying agents, lubricants, glidants, emollients, humectants, thickeners, flavoring agents, preservatives, or pH buffers. Examples of suitable buffers include phosphate buffers such as phosphate buffered saline (PBS), acetate buffers, benzoate buffers, citrate buffers, lactate buffers, maleate buffers, and tartarate buffers. Buffered carriers like Hanks's solution, Ringer's solution, dextrose solution, 5% human serum albumin, Ringer's dextrose, dextrose and sodium chloride, lactated Ringer's and fixed oils, polyethylene glycol, polyvinyl pyrrolidone, or lecithin can be used. Monoethanolamine, diethanolamine, tromethamine, and glycine solutions can also be used as suitable buffers. Liposomes and nonaqueous vehicles such as fixed oils may also be used as carriers. Further examples of suitable carriers are described in “Remington's Pharmaceutical Sciences” by E. W. Martin. One of skill in the art can select the formulation to suit the mode of administration.

The composition can be processed and purified by a number of procedures before being administered to a subject. Optionally, the composition can be screened, separated, stored, extracted, blended, and/or purified before use. Additional steps as known to those of skill in the art can also be used to purify the composition. The composition can be further processed into particular types of dosage or delivery vehicles. Optionally, the composition can be processed as a nutraceutical composition or a dietary supplement. Optionally, the composition can be processed as a nutritional drink or beverage, animal feed, pet food, or olive oil. Optionally, the composition is in the form of a beverage, nutritional bar, food additive, tablet, pill, granule, capsule, satchet, or effervescent formulation.

The compositions described herein are stable at a temperature at about ambient temperature for a period of time and are thus suitable for storage. As used herein, ambient temperature refers to room temperature (e.g., from 68° F. to 77° F.). In some examples, the compositions are stable for at least three months, at least four months, at least five months, at least six months, at least seven months, at least eight months, at least nine months, at least ten months, at least eleven months, at least twelve months, at least thirteen months, at least fourteen months, at least fifteen months, at least sixteen months, at least seventeen months, at least eighteen months, or greater.

Optionally, the stability of the composition can be determined by measuring the oleuropein content in the composition before storage and after storage. In the compositions described herein, at least 80% of the oleuropein in the olive extract is present after twelve months at ambient temperature. For example, at least 85% or at least 90% of the oleuropein present in the olive extract before storage is present after twelve months at ambient temperature.

II. Dosage Formulations

The compositions can be administered to a subject in an amount sufficient to produce the desired result as described herein (e.g., an amount sufficient to promote joint health in a subject). The olive extract can be administered to the subject in an amount of from 1 mg to 2000 mg per day (e.g., from 2 mg to 1750 mg per day, from 5 mg to 1500 mg per day, from 10 mg to 1000 mg per day, or from 25 mg to 500 mg per day). Hydroxytyrosol can be administered to the subject in an amount of from 3 mg to 50 mg per day (e.g., from 5 mg to 45 mg, from 10 mg to 40 mg, or from 20 mg to 30 mg). Oleuropein can be administered to the subject in an amount of from 1 mg to 50 mg per day (e.g., from 5 mg to 45 mg, from 10 mg to 40 mg, or from 20 mg to 30 mg). Tyrosol can be administered to the subject in an amount of from 2 mg to 30 mg per day (e.g., from 5 mg to 25 mg or from 10 mg to 20 mg). Quercetin can be administered to the subject in an amount of from 10 mg to 3,000 mg per day (e.g., from 50 mg to 2,500 mg, from 100 mg to 2,000 mg, from 500 mg to 1,500 mg, or from 600 mg to 1,200 mg). Red onion powder or extract can be administered in an amount of from 100 mg to 2,000 mg per day (e.g., from 500 mg to 1,500 mg or from 750 mg to 1,000 mg). Naringin can be administered to the subject in an amount of from 5 mg to 100 mg per day (e.g., from 10 mg to 90 mg, from 25 mg to 75 mg, or from 30 mg to 70 mg). Sinomenine can be administered to the subject in an amount of from 5 mg to 2,000 mg per day (e.g., from 50 mg to 1,500 mg, from 75 mg to 1,250 mg, from 100 mg to 1,000 mg, or from 250 mg to 750 mg). Piperine can be administered to the subject in an amount of from 5 mg to 50 mg per day (e.g., from 10 mg to 45 mg, from 15 mg to 40 mg, or from 20 mg to 35 mg). Glycyrrhizin can be administered to the subject in an amount of from 5 mg to 1,000 mg per day (e.g., from 10 mg to 900 mg, from 50 mg to 800 mg, from 75 mg to 700 mg, from 100 mg to 600 mg, or from 200 mg to 500 mg). A nitrile glycoside can be administered in an amount of from 5 mg to 2,000 mg per day (e.g., from 10 mg to 1,750 mg, from 50 mg to 1,500 mg, from 75 mg to 1,250 mg, from 100 mg to 1,000 mg, or from 250 mg to 750 mg). Cuminum cymium can be administered to the subject in an amount of from 5 mg to 2,000 mg per day (e.g., from 10 mg to 1,750 mg, from 50 mg to 1,500 mg, from 75 mg to 1,250 mg, from 100 mg to 1,000 mg, or from 250 mg to 750 mg). Curcumin can be administered to the subject in an amount of from 3 mg to 50 mg per day (e.g., from 5 mg to 45 mg, from 10 mg to 40 mg, or from 20 mg to 30 mg). Vitamin D can be administered to the subject in an amount of from 500 iu to 5,000 iu per day (e.g., from 750 iu to 4,000 iu, from 1,000 iu to 3,000 iu, or from 1,500 iu to 2,500 iu). Willow bark extract can be administered to the subject in an amount of from 100 mg to 2,000 mg per day (e.g., from 500 mg to 1,500 mg, from 750 to 1,250 mg, or from 800 mg to 1,000 mg). Caffeine can be administered to the subject in an amount of from 25 mg to 200 mg per day (e.g., from 50 mg to 150 mg or from 75 mg to 125 mg). A hydrogen carbonate can be administered to the subject in an amount of from 5 mg to 2,000 mg per day (e.g., from 10 mg to 1,750 mg, from 50 mg to 1,500 mg, from 75 mg to 1,250 mg, from 100 mg to 1,000 mg, or from 250 mg to 750 mg).

Optionally, the composition is administered orally. Optionally, the composition is administered to the subject at least once per day or up to throughout the day on one day or on consecutive days, for a period of time. An effective amount of the composition will be determined on an individual basis. The dosage may be based, at least in part, on the particular desired effect, on the individual's size, age, and gender, on the physical and health characteristics of the subject, and/or on the type and severity of joint discomfort or joint use. For example, a runner or hiker may have excessive use of joints and be in need of a greater amount of the composition.

Optionally, the subject can be placed on a dosage regimen for administering the above-described composition. The dosage regimen can include administering the composition on its own or as a supplement to a meal. Additionally, the dosage regimen can include administering the composition once a day or multiple times a day in order to meet the required dosage. Optionally, the dosage regimen includes administering a single dose to provide the subject with the total required daily amount of the composition. Optionally, the total dosage of the composition is from 0.005 to 10 grams per day (e.g., approximately 100 mg per day).

III. Methods of Use

Also described herein are methods for maintaining, supporting, and/or improving the health and comfort of a subject (e.g., joint health, energy, cardiovascular health, and immune health). The methods of maintaining, supporting, and/or improving health and comfort in a subject comprise selecting a healthy subject and administering to the subject a composition as described herein. As used herein, a healthy subject refers to a subject that has never had or does not presently have a diagnosed diseased symptom (e.g., an arthropathic symptom) and/or a subject that is asymptomatic of a disease pathology. For example, the healthy subject can be a non-arthropathic subject (i.e., a subject not exhibiting any arthropathic symptoms nor suffering from any arthropathic conditions). Exemplary arthropathic conditions include rheumatoid arthritis, rheumatic fever, osteoarthritis, spondylarthropathy, ankylosing spondylitis, Reiter's syndrome, psoriatic arthropathy, juvenile arthropathy, reactive arthropathy, enteropathic arthropathy, crystal arthropathy (e.g., gout), diabetic arthropathy, and neuropathic arthropathy.

Optionally, the method further comprises administering to the subject a second compound or composition. The second compound or composition can include an anti-arthritic or analgesic compound or composition (e.g., an anti-inflammatory, aspirin, ibuprofen, acetaminophen, or naproxen), a nutraceutical additive, vitamin, mineral, or an herbal extract. The second compound or composition and the compositions described herein can be administered in any order, including simultaneous administration, as well as temporally spaced order of up to several days apart. The methods may also include more than a single administration of the second compound or composition and/or the compositions described herein. The administration of the second compound or composition and the compositions described herein may be by the same or different routes and concurrently or sequentially.

Further described herein is a use of a composition as described herein for maintaining and supporting the health and comfort of a subject (e.g., joint health, energy, cardiovascular health, and immune health).

There compositions described herein can be administered in any therapeutically acceptable manner of administering a dietary supplement, medical food, or drug. Optionally, the composition can be administered as a tablet, capsule, pill, powder, liquid, powder mixed with a liquid, drink, topical cream, gel, ointment, emulsion and/or paste. In these forms, the method of administering the composition can include orally ingesting or topically applying.

As used herein, references to promoting and improving include a change of 5%, 10%, 20%, 30%, 40%, 50%, 60%, 70%, 80%, 90%, or greater in health (e.g., quality of life) as compared to a control level. Parameters of quality of life include walking, stair climbing, gripping, and other daily activities. As used herein, control refers to the untreated condition. Such terms can include, but do not necessarily include, complete prevention.

As used herein, subject means both mammals and non-mammals. Mammals include, for example, humans; non-human primates, e.g., apes and monkeys; cattle; horses; dogs; cats; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1

### Exemplary Composition

An exemplary combination of olive extract components in a stable composition is shown in Table 1.

TABLE 1

| Component | Daily Dosage |
|---|---|
| Hydroxytyrosol | 3 to 50 mg |
| Oleuropein | 1 mg to 30 mg |
| Tyrosol | 1 to 30 mg |

### Example 2

### Stability Testing

While one polyphenol can be stable when alone, a method has not been documented to stabilize the three polyphenols together in one composition. Multiple studies have shown that when hydroxytyrosol, tyrosol, and oleuropein are combined, the polyphenols exhibit significant instability, showing a decrease of 70% over one year. The studies also show that oleuropein converts to hydroxytyrosol at a very fast rate. See Baiano et al., “Changes in phenolic content and antioxidant activity of Italian extra-virgin olive oils during storage,” 74(2): C177-83 (March 2009); Adrover et al., “Influence of storage of extra virgin olive oil on physicochemical and sensory parameters;” and Cinquanta et al., “Oxidative stability of virgin olive oils,” JAOCS, 78(12): 1197-1202 (2001). The method of extend the shelf life of all three polyphenols in one composition as described herein is a unique finding. As described herein, when hydroxytyrosol, tyrosol, and oleuropein ratios are in a specific range, the shelf lives of all three polyphenols are dramatically increased.

Composition 1 was prepared containing 10.24% hydroxytyrosol, 3.94% tyrosol, and 3.31% oleuropein, which provided a 3.09:1 ratio of hydroxytyrosol to oleuropein. After storing at room temperature for 306 days, the composition contained 14.22% hydroxytyrosol, 3.42% of tyrosol, and 1.76% of oleuropein. See Table 2. Thus, the amount of oleuropein decreased more than 50% when the original composition was prepared with a 3:09:1 ratio of hydroxytyrosol to oleuropein.

TABLE 2

| | Hydroxytyrosol | Tyrosol | Oleuropein |
|---|---|---|---|
| Day 1 | 10.24% | 3.94% | 3.31% |
| Day 306 | 14.22% | 3.42% | 1.76% |

Composition 2 was prepared containing 10.53% hydroxytyrosol, 4.12% tyrosol, and 4.02% oleuropein, which provided a 2.62:1 ratio of hydroxytyrosol to oleuropein. After storing at room temperature for 369 days, the composition contained 14.2% of hydroxytyrosol, 4.4% of tyrosol, and 4.1% of oleuropein. See Table 3. The amount of oleuropein did not decrease when the original composition was prepared with a 2.61:1 ratio of hydroxytyrosol to oleuropein.

TABLE 3

| | Hydroxytyrosol | Tyrosol | Oleuropein |
|---|---|---|---|
| Day 1 | 10.53% | 4.12% | 4.02% |
| Day 369 | 14.2% | 4.4% | 4.1% |

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus a combination of steps, elements, components, or constituents may be explicitly mentioned herein; however, all other combinations of steps, elements, components, and constituents are included, even though not explicitly stated.

What is claimed is:

1. A composition, comprising:
an olive extract comprising hydroxytyrosol, tyrosol, and oleuropein, wherein the weight ratio of hydroxytyrosol to oleuropein is 3:1 or less; and wherein the weight ratio of hydroxytyrosol to tyrosol is 2.85:1 or less,
wherein the composition is stable for at least twelve months at ambient temperature.

2. The composition of claim 1, wherein at least 80% of the oleuropein in the olive extract is present after twelve months at ambient temperature.

3. The composition of claim 1, wherein at least 85% of the oleuropein in the olive extract is present after twelve months at ambient temperature.

4. The composition of claim 1, wherein at least 90% of the oleuropein in the olive extract is present after twelve months at ambient temperature.

5. The composition of claim 1, further comprising at least one additional compound selected from the group consisting of extracts of grapes, quercetin, red onion powder or extract, naringin, sinomenine, piperine, glycyrrhizin, a nitrile glycoside, cuminum cyminum, curcumin, vitamin D, willow bark extract, theobromine, a coffee extract, and a hydrogen carbonate.

6. The composition of claim 1, wherein the weight ratio of hydroxytyrosol to tyrosol is from 1:1 to 2.85:1.

7. The composition of claim 1, wherein the weight ratio of hydroxytyrosol to tyrosol is from 1.5:1 to 2.75:1.

8. The composition of claim 1, wherein the weight ratio of hydroxytyrosol to tyrosol is from 2:1 to 2.7:1.

9. The composition of claim 1, wherein the weight ratio of hydroxytyrosol to oleuropein is from 1:1 to 3:1.

10. The composition of claim 1, wherein the weight ratio of hydroxytyrosol to oleuropein is from 1.5:1 to 2.8:1.

11. The composition of claim 1, wherein the weight ratio of hydroxytyrosol to oleuropein is from 2:1 to 2.7:1.

12. The composition of claim 1, wherein the weight ratio of tyrosol to oleuropein is from 1:1 to 2.5:1.

13. The composition of claim 1, wherein the weight ratio of tyrosol to oleuropein is from 1:1 to 2:1.

14. The composition of claim 1, wherein the olive extract is a powder or a liquid.

15. The composition of claim 1, wherein the composition is a nutraceutical composition or a dietary supplement.

16. The composition of claim 1, wherein the composition is in the form of a liquid, a nutritional bar, food additive, tablet, pill, oil, granule, capsule, or effervescent formulation.

17. A method of maintaining health and comfort in a subject, comprising:
selecting a healthy subject; and
administering to the subject a composition of claim 1.

18. The method of claim 17, wherein the healthy subject is a non-arthritic subject or a non-diseased person.

19. The method of claim 17, further comprising administering to the subject a second compound or composition, wherein the second compound or composition includes a nutraceutical additive, a vitamin, a mineral, or an herbal extract.

20. A dietary supplement, comprising:
an olive extract comprising hydroxytyrosol, tyrosol, and oleuropein, wherein the weight ratio of hydroxytyrosol to oleuropein is 3:1 or less; and wherein the weight ratio of hydroxytyrosol to tyrosol is 2.85:1 or less,
wherein the composition is stable for at least twelve months at ambient temperature.
